(19) United States
(12) Patent Application Publication
(10) Pub. No.: US 2020/0277646 A1
(43) Pub. Date: Sep. 3, 2020

(54) METHOD OF DETERMINING TERPENE-BASED BIOCONTROL AGENTS FOR CANNABIS AND HEMP

(71) Applicants: Cindy Orser, Las Vegas, NV (US); Aaron Hilyard, Las Vegas, NV (US)

(72) Inventors: Cindy Orser, Las Vegas, NV (US); Aaron Hilyard, Las Vegas, NV (US)

(21) Appl. No.: 16/658,154

(22) Filed: Oct. 20, 2019

Related U.S. Application Data
(60) Provisional application No. 62/812,992, filed on Mar. 2, 2019.

(51) Int. Cl. C12Q 1/18; C12Q 1/04

(52) U.S. Cl. CPC C12Q 1/18; C12Q 1/04

(57) ABSTRACT

The embodiments disclose a method of determining terpene-based biocontrol agents for cannabis and hemp plants including placing terpene isolates-soaked discs in a pattern onto the bacterial inoculated plates in duplicates with a diluent-soaked negative control disc in the center, testing and evaluating combinations of bioactive terpene isolates identified under specific aims for synergy, wherein the specific aims are to evaluate individual terpenes for effectiveness in controlling common fungal and bacterial populations found on indoor and outdoor grown cannabis plants, developing terpene isolates formulations and retest under the specific aims to ensure that added ingredients do not interfere or dampen bioactivity, testing terpene isolates formulations on cannabis and hemp seedlings, harvested cannabis and hemp flowers and cured cannabis and hemp flower for suitability, and testing terpene isolates formulations in challenge experiments on cannabis and hemp seedlings.

### CROSS-REFERENCED TO RELATED APPLICATIONS

This application is based on U.S. Provisional Patent Application Ser. No. 62/812,992 filed Mar. 2, 2019, entitled “A METHOD OF DETERMINING TERPENE-BASED BIOCONTROL AGENTS FOR CANNABIS AND HEMP”, by CINDY ORSER.

### BACKGROUND

Mold presents a difficult problem for many cannabis cultivators, resulting in about 15% failure rate for flower. Mold infections increase cultivators cost and present potential health risks for consumers. Synthetic fungicides also present their own regulatory, economic, and health risks.

### BRIEF DESCRIPTION OF THE DRAWINGS

FIG. 1 shows for illustrative purposes only an example of placing terpene-soaked discs in a pattern onto bacterial inoculated plates of one embodiment.

FIG. 2 shows for illustrative purposes only an example of terpene concentrations plot analysis of terpenoid data of one embodiment.

FIG. 3 shows for illustrative purposes only an example of profiled chemicals of one embodiment.

FIG. 4 shows for illustrative purposes only an example of mean terpenoid content test results of one embodiment.

FIG. 5 shows for illustrative purposes only an example of a method of evaluating individual terpenes for effectiveness of one embodiment.

FIG. 6 shows for illustrative purposes only an example of evaluation results #1 of individual terpenes for effectiveness of one embodiment.

FIG. 7 shows for illustrative purposes only an example of evaluation results #2 of individual terpenes for effectiveness of one embodiment.

FIG. 8 shows for illustrative purposes only an example of terpinolene concentrations treatment sample culture plates of one embodiment.

FIG. 9 shows for illustrative purposes only an example of cultured yeast and mold species from certified reference materials of one embodiment.

FIG. 10 shows for illustrative purposes only an example of several microbial species cultures of one embodiment.

FIG. 11A shows for illustrative purposes only an example of group 1 terpenes selected for trials across several microbial species of one embodiment.

FIG. 11B shows for illustrative purposes only an example of group 2 terpenes selected for trials across several microbial species of one embodiment.

FIG. 12A shows for illustrative purposes only an example of cultures isolated from cannabis flower of one embodiment.

FIG. 12B shows for illustrative purposes only an example of all treated samples shows less growth of one embodiment.

FIG. 13 shows a block diagram of an overview of remediation by terpene treatment of one embodiment.

FIG. 14 shows a block diagram of an overview of a tabulation of remediation by terpene treatment mold growth analysis results of one embodiment.

FIG. 15 shows for illustrative purposes only an example of images from untreated and treated cultures remediation samples of one embodiment.

FIG. 16 shows for illustrative purposes only an example of a digital inhibition evaluation device of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview:

It should be noted that the descriptions that follow, for example, in terms of a method of determining terpene-based biocontrol agents for cannabis and hemp is described for illustrative purposes and the underlying system can apply to any number and multiple types terpene isolates. In one embodiment of the present invention, the method of determining terpene-based biocontrol agents for use on cannabis and hemp can be configured using plant terpene isolates derived essential oils. The method of determining terpene-based biocontrol agents for use on cannabis and hemp can be configured to include multiple cultivars testing and can be configured to include using individual terpene isolates at various dilutions using the embodiments.

### Terpenes as Natural Fungicides:

Terpenes are major components of plant derived essential oils used by humans for thousands of years, and they are abundant in unique combinations in cannabis. Terpenes can be used to distinguish cultivars from one another, many terpenes have documented medical effects in humans, and terpenes also have a natural role in the plant's interaction with its environment. Data from flower samples collected in Nevada in 2018 show that certain cultivars fail for mold much more often than others.

We consider several possible explanations for higher mold failure rates on certain cultivars and discuss some basic agricultural considerations that can impact the microbiological environment including currently available commercial fungicides derived from plant sources. Addition of individual terpene isolates at various dilutions showed decreased mold growth versus a control when added to failing client samples. This indicates that some terpenes may inhibit mold species present in the commercial environment. And the antifungal effect of each terpene addition was also related to the natural terpene profile of each particular cultivar.

Terpenes are major components of plant derived essential oils and have been used by humans for thousands of years. Terpenes play a natural role in a plant's interaction with its environment including repelling pests and attracting pollinators. Mold presents a difficult problem for many cannabis cultivators, resulting in about 15% failure rate for flower. Mold infections increase cultivators cost and present potential health risks for consumers. Synthetic fungicides also present their own regulatory, economic, and health risks. The production of terpenes by cannabis plants are used as naturally-derived microbial control agents. Not only can terpenes be used to distinguish and categorize cannabis cultivars from one another, we noticed that certain cultivars fail for mold much more often than others and it can be tied to their terpene chemoprofile. The use of prophylactically applied terpenes as natural biocontrol agents has the added benefit of augmenting the resulting terpene content in the dried flower and products made from the flower including extracts, imbuing added benefits to the cannabis user.

This preliminary data suggests that individual terpenes or terpene mixes may be effective as fungicides and broad spectrum biocides during cultivation, and that effective terpene mixes may be optimized for different cultivars. Cannabis derived terpene fungicides and biocides may also be effective on other crops. As an aside I see a secondary benefit to the use of terpenes for biocontrol on cannabis which is that the terpene content of the resulting crop can/could be influenced in a positive way since terpenes are bioactive molecules in mammals as well.

Cannabis terpene-mimickry in beer to define terpene profiles in hops and options during and after the beer brewing process to influence the resulting terpene profile contribution for aroma, flavor, and specific physiological outcomes in the end user including but not limited to cannabis strain specific terpene-mimickry.

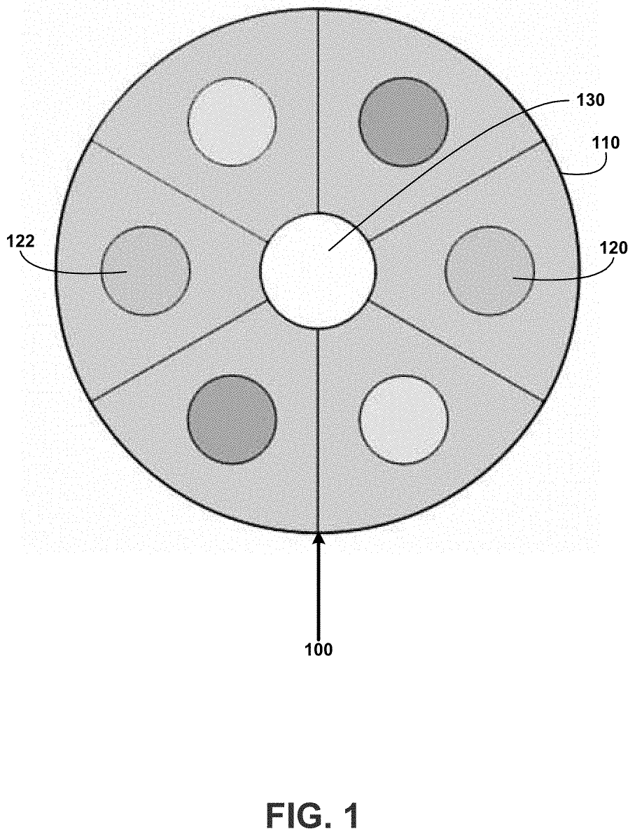

FIG. 1 shows for illustrative purposes only an example of placing terpene-soaked discs in a pattern onto bacterial inoculated plates of one embodiment. FIG. 1 shows an example of a method of determining terpene-based biocontrol agents for cannabis and hemp by placing terpene-soaked discs in a pattern onto the bacterial inoculated plates in duplicates with a diluent-soaked negative control disc in the center 100. FIG. 1 shows an example a bacterial inoculated plate 110 with a diluent-soaked negative control disc 130 in the center and terpene-soaked disc #1 120 and a terpene-soaked disc #1 duplicate 122. This prepares the samples for testing of the efficacy of a terpene fungicidal and broad spectrum microbiocidal function in controlling mold on various cannabis plants.

Terpenes are the Next Frontier in therapeutic impacts. Current thought is that there is no end to the phytocannabinoid pharmacological revelations from a highly effective pain management alternative to a non-toxic anti-cancer remedy of cannabidiol (CBD) and tetrahy drocannabinol (THC), the two powerhouse cannabinoids respectively. However, the phytotherapeutic story is shifting to what will be a dramatic ascendency of terpenes and their therapeutic impact either individually or in concert with THC and/or CBD. Terpenes heretofore have been used predominantly as flavorings and fragrances common in our diet, and are considered GRAS compounds by the US Federal and Drug Administration and have no controlled substance restrictions associated with phytocannabinoids.

It turns out that terpenes are affecting us much more than we might think. As examples: limonene is a strong anxiolytic, is active against bacteria associated with acnel and can induce the suicide of cancer cells. Another terpene beta-caryophyllene is a nonpsychoactive anti-inflammatory via CB2 binding. Another beta-myrcene is a sedating muscle relaxant anti-inflammatory. Yet another terpene alpha-pinene, the most abundant terpene among all plants is an antimicrobial and more importantly acts as an acetylcholinesterase inhibitor which may aid in memory loss associated with several human neurological diseases.

The rising eminence of terpenes couldn't be coming at a better time for the cannabis industry that needs a strategy for demarcating medical marijuana from recreational marijuana. Today, terpenes constitute the medicinal subtlety of the entourage effect that we all keep hearing about. There are probably more biopharmaceutical messengers yet to be revealed by this most extraordinary plant, Cannabis sativa L.

Few cannabis complicit states even require testing and reporting of terpenes. Most cannabis consumers remain in the dark about the importance of terpenes. The State of Nevada was the first state to have the foresight to require reporting of 11 terpenes as a component of potency.

Unfortunately, many cannabis extract producers are unaware that their procedures are basically throwing away the terpenes to the atmosphere rather than bringing them along with the two powerhouses, THC and CBD, which remain the coveted measure of wholesale valuation. Clearly, retaining the entire terpene complement going forward appears to be worth the effort during cannabis extraction.

Not surprisingly, it was Ethan Russo's review article in 2011 that portended the current therapeutic extent of cannabis-derived terpenes on human health and behavior. Remarkably, terpenes are potent even when inhaled in ambient air and can subsequently be detected at quantifiable levels in your serum.

Terpenes and cannabinoids (also chemically known as phenolic terpenes), share a common biochemical precursor, geranyl pyrophosphate and over 10,000 terpenes have been identified, albeit their biochemical and genetic pathways are not completely resolved. Another consequence of the focused spotlight on THC and CBD.

Terpenes act remarkably in concert with phytocannabinoids and in particular to diminish the intoxicating effects of THC. From the MMJ side of the industry, if the intoxicating effect of THC can be controlled, reduced or eliminated while maintaining its therapeutic merits, the application of cannabis derived products could be expanded dramatically. Terpenes can make up to 10% of trichome content but are subject to loss from processing and subsequent drying and storage. Terpenes come in different formats with the three monoterpenes, limonene, alpha-pinene and beta-myrcene dominating, followed by sesquiterpenes, like beta-caryophyllene and alpha-humulene. Importantly, just like cannabinoids, terpene synthesis is under genetic control.

From a pharmacological standpoint, terpenes are highly evolved lipophilic molecules that can interact with other lipophilic structures like membranes and the many embedded receptors that reside there, resulting in neurological responses, the detection of odors, downstream enzymatic changes or acting as a THC antidote. Terpenes' lipophilic (hydrophobic) nature also gives them the unique ability to permeate skin. Remarkably, the use of terpenes as facilitating permeants in topical formulations not involving cannabinoids was reported 13 years ago.

Topically applied drugs must somehow permeate the stratum corneum or the outermost layer of epidermis, layers of dead cells embedded in a lipid matrix. Topicals passing through the stratum corneum must either move into cells (intracellularly) or move between cells (intercellularly) and the lipophilicity of the compound formulation is the key. Cornwell et al. found that formulations with high levels of specific terpenes could increase permeation by up to a 1,000-fold with the intercellular concentration of limonene, cineole and nerolidol reaching 40-60% of the tissue treated with neat terpenes. Plants are clearly better remedies than the extracts made from them and stand apart from the empty promise of modern pharmaceuticals. Going forward, more attention needs to be focused on the contribution of terpenes both individually and in combination with phytocannabinoids.

Other articles discuss the terpenes including: 1 Kim et al. (2008) Biological activity of Korean Citrus obovoides essential oils against acne-inducing bacteria. Biosci Biotechnol Biochem 72:2507-2513. 2 Vigushin et al. (1998) Phase I and pharmacokinetic study of d-limonene in patients with advanced cancer. Cancer Chemother Pharmacol 42:111-117. 3 Gertsch et al. (2008) Beta-caryophyllene is a dietary cannabinoid. PNAS USA 105:9099-9104. 4 do Vale et al. (2002) Central effects of citral, myrcene and limonene, constituents of essential oil chemotype from Lippia alba (Mill.) n.e. Brown. Phytomed 9:709-714. 5 Perry et al. (2000) in vitro inhibition of human erythrocyte acetylcholinesterase by Salvia lavandulaefolia essential oil and constituent terpenes. J Pharm Parmacolo 52:895-902. 6 Russo E. (2011) Taming THC: potential cannabis synergy and phytocannabinoid-terpenoid entourage effects. British J Pharmacology 163:1344-1364 Wednesday, Jun. 7, 2017. 7 Ibid. 8 Langenheim JH (1994) higher plant terpenoids: a phytocentric overview of their ecological roles. J Chem Ecol 20:1223-1279. 9 Cornwell PA, Barry BW (1994) Sesquiterpene components of volatile oils as skin penetration enhancers for the hydrophilic permeant 5-fluorouracil. J Pharm Pharmacol 46:261-269. 10 Moghimi et al. (1998) Enhancement by terpenes of 5-fluorouracil permeation through the stratum corneum: model solvent approach. J Pharm Pharmacol 50: 955-964 of one embodiment.

### Detailed Description:

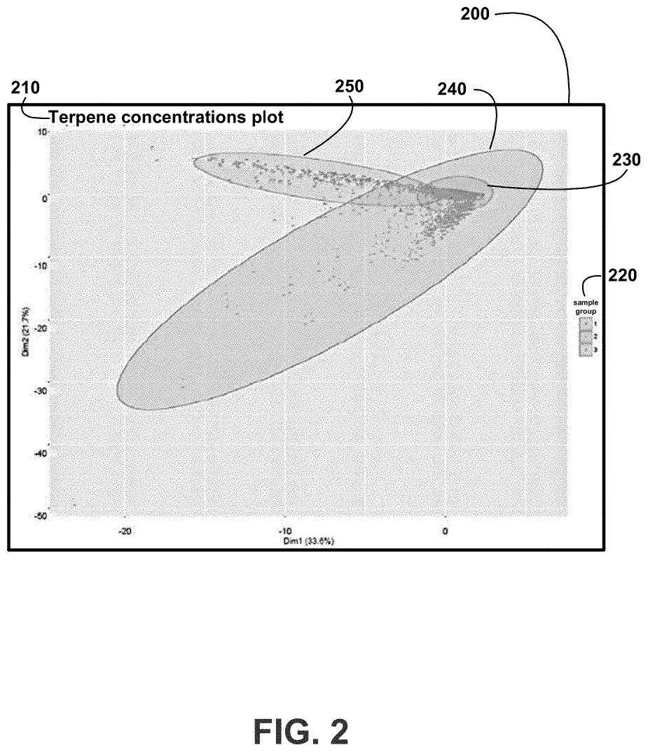

FIG. 2 shows for illustrative purposes only an example of terpene concentrations plot analysis of terpenoid data of one embodiment. FIG. 2A shows an example of a terpene concentrations plot analysis of terpenoid data 200. The graphical display of the terpene concentrations plot 210 of the terpenoid data clearly shows the effective distribution of terpene sample tests. This method of analysis aids researchers in a arriving at a clear understanding of the evaluations and helps them to direct their attention to the most effective applications of one embodiment.

An unintended consequence of state-mandated cannabis testing regulations has been the resulting database from the analysis of thousands of individual cannabis flower samples from artificially restricted geographical regions. The resulting detailed chemical database can serve as the basis for the development of a chemotaxonomic classification scheme outside of conjectural cultivar naming by strain. Chemotaxonomic classification schemes for cannabis cultivars have previously been reported by others based largely on cannabis strains grown in California under an unregulated testing environment or in Europe from strains grown by a single cultivator. In this study 2,237 individual cannabis flower samples, representing 204 individual strains across 27 cultivators in a tightly regulated Nevada cannabis testing market, were analyzed across 11 cannabinoids and 19 terpenoids.

Even though 98.3% of the samples were from drug Type I cannabis strains by cannabidiolic acid/tetrahydrocannabinolic acid (THCA) ratio of <0.5 CBDA, principal component analysis (PCA) of the combined dataset resulted in three distinct sample group 220 of data that were distinguishable by terpene profiles alone. Sample group #1 230, sample group #2 240, and sample group #3 250 show the data results of three different terpene profiles. Further dissection of individual strains by cultivators within samples revealed striking fidelity of terpenoid profiles and also revealed a few outliers. We propose that three terpenoid sample assignments account for the diversity of drug type cannabis strains currently being grown in Nevada of one embodiment.

### Profiled Chemicals:

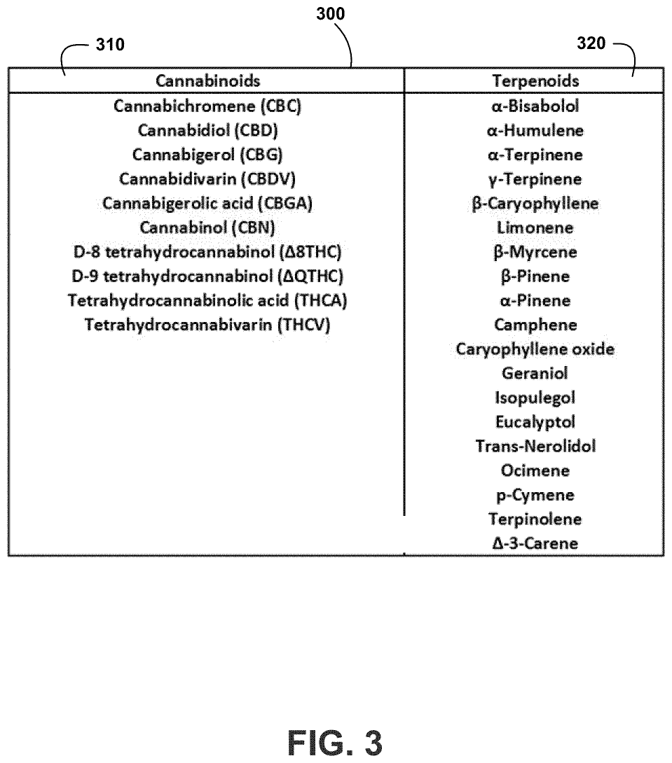

FIG. 3 shows for illustrative purposes only an example of profiled chemicals of one embodiment. FIG. 3 shows examples of profiled chemicals 300 including a listing of examples of cannabinoids 310 and terpenoids 320. The objective is to identify unique combinations of plant-derived terpenes for use as prophylactic applications on cannabis plants to control either fungal or bacterial populations and/or mites and aphids as 100% natural, non-toxic plant-powered “terpenated pest control” with the added benefit of terpo-charging the resulting cannabis crop.

### Mean Terpenoid Content Test Results:

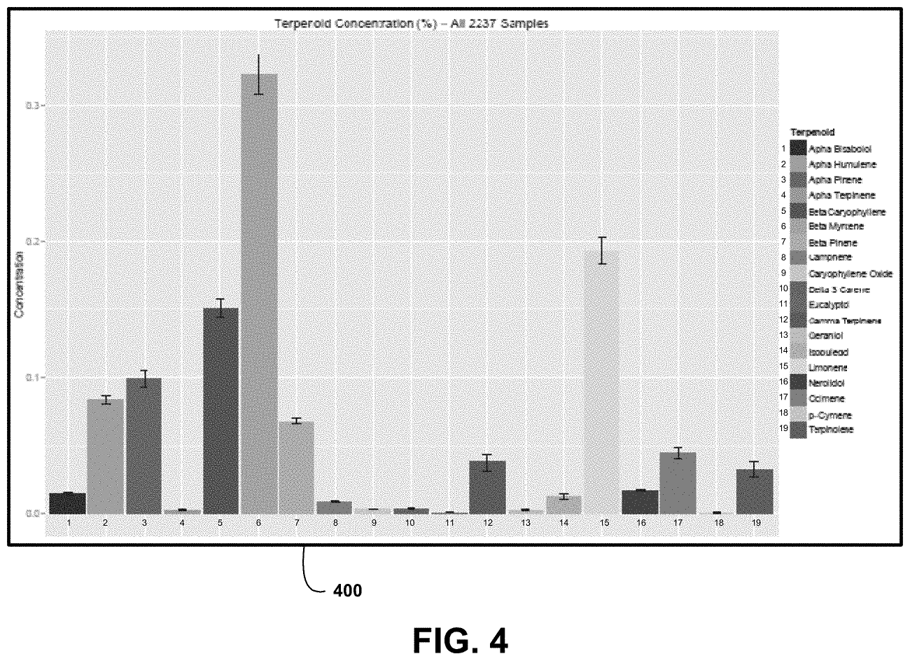

FIG. 4 shows for illustrative purposes only an example of mean terpenoid content test results of one embodiment. FIG. 4 shows a bar chart of mean terpenoid content test results 400. The lack of horticultural or agronomic naming conventions and registrations in the cannabis industry has created a confusing collection of strain names, making authenticity questionable for the cannabis consumer. Various efforts have been underway to make sense of cannabis strain names through the use of data analytics on broader cannabis flower chemoprofiles beyond strictly tetrahydrocannabinol (THC) and cannabidiol (CBD) potency even though harvesting a consistently reproducible cannabis crop is challenging under the best of agronomic standards.

There are many environmental factors besides genetics that can impact the resulting chemical content of an agronomic plant. Reports indicate that cannabinoid and terpenoid content varies both intra-plant, inter-plant and between harvest lots. Reducing consistently-grown cannabis to its extract is the best approach to achieving chemical profile uniformity. Cannabis drug-type plants that have a cannabidiolic acid (CBDA)/tetrahydrocannabinolic acid (THCA) ratio of <0.5 are referred to as drug type I or broad leaflet drug-type (BLDT), cannabis plants with a CBDA/THCA ratio between 0.5 and 3.0 are type II or narrow leaflet drug-type (NLDT) and those plants with a CBDA/THCA ratio of >3.0 are type III or hemp.

The obsession surrounding cannabinoids and in particular (−) trans-Δ9-tetrahydrocannabinol (−Δ9-THC) and cannabidiol (CBD) content in cannabis cultivars has overshadowed the importance of the terpenoid profile and content in specific cannabis cultivars. Today we know that terpenoids are contributing pharmacologically active compounds in cannabis and can be used to distinguish cannabis cultivars. Of the roughly 140 identified terpenoids in cannabis, there seems to be a consensus in the literature that range between 17 to 19 are the most useful in defining a cannabis chemotype.

Terpenoid content in the cured flower can typically range from 0.5% to 3%. Terpenoids demonstrate effects on the brain at very low ambient air levels in animal studies. Generally speaking terpenoids contribute a sedative and anxiolytic effect to cannabis with more specific pharmacological effects attributed to individual terpenoids; such as, pinene exhibiting antibiotic activity [14] or β-caryophyllene providing gastric cytoprotective activity [15] or the anticonvulsive effects of β-myrcene.

Previous groups have made great strides in demonstrating that terpenoid content can be used to distinguish cannabis strains, varieties or cultivars based on the nomenclature in use. These studies have also highlighted the importance of obtaining cannabis samples of sufficient size and representativeness to result in a valid test. Notably, previous reports demonstrate the need for the use of validated analytical test methods as well as reproducibility of sample set data.

In 2010, Fishedick et al. showed that across 11 cannabis varieties, all grown by one cultivator, each variety was distinguishable based on the occurrence of 36 different chemical compounds including 27 terpenoids using principle component analysis (PCA). A recent genomic analysis of diversity among 340 Cannabis sativa L. varieties demonstrated the existence of three major cannabis groups represented by BLDT, NLDT and hemp [21]. In an elegant time course study over the growth cycle of specific chemotyped cannabis plants Aizpurua-Olaizola et al. [1] found differences in the evolution of monoterpene and sesquiterpene patterns within chemotypes. Here we report on the analysis of the chemoprofile data for 2,237 individual cannabis flower samples representing 204 individual cultivars across 27 cultivators in a tightly regulated Nevada cannabis testing market. Even though all of the samples except for 1.7% of the samples were type I based on CBDA/THCA ratio, the terpenoid chemoprofiles distinguished the samples into three separate samples of one embodiment.

### A Method of Evaluating Individual Terpenes for Effectiveness:

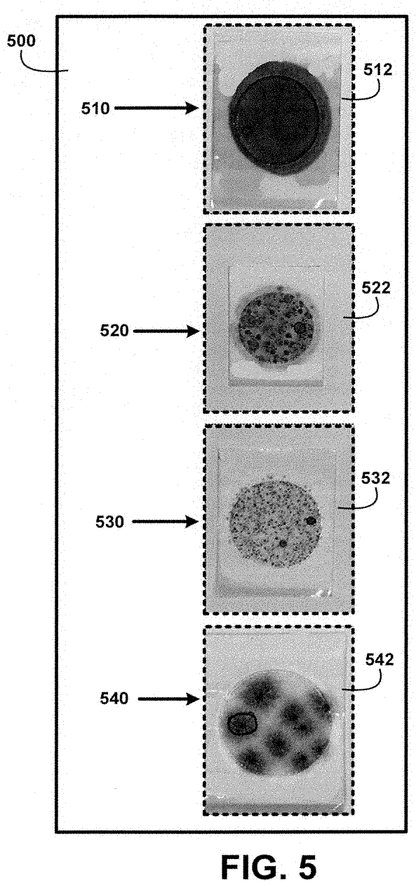

FIG. 5 shows for illustrative purposes only an example of a method of evaluating individual terpenes for effectiveness of one embodiment. FIG. 5 shows a method of evaluating individual terpenes for effectiveness in controlling common fungal and bacterial populations. The method includes one sample in an EB Plate that is a control sample. Mold Plate 1, Mold Plate 2, and Mold Plate 3 are samples of terpene-soaked discs in a pattern onto bacterial inoculated plates as described in FIG. 1. Each of the Plates represents a terpene-soaked disc that is placed in the petri dish in the pattern shown in FIG. 1. The specific aims are to evaluate individual terpenes for effectiveness in controlling common fungal and bacterial populations found on indoor and outdoor grown cannabis plants.

At least one device for mixing formulated individual terpene isolates concentrations and for mixing formulated combinations of individual terpene isolates concentrations. At least one device for applying at least one of the mixed terpene formulations on cannabis seedlings and plants is used for field testing terpene inhibition treatments. An infrared camera with Wi-Fi and cellular connectivity coupled electronically to a digital server using cellular connectivity captures time-stamped images at intervals of microbial species infected cannabis seedlings and plants growing indoors or outdoors that have been treated with at least one application of at least one embodiment of a terpene isolates concentration. The infrared images detect the areas of the microbial infections and record on server databases the progress of the inhibition of the microbial growth.

The server scanner with OCR converts the time-stamped data into digital data that is recorded with the infrared images on the digital server database. An algorithm of the digital server computer measures the infected surface areas and records those measurements on the digital server database. The computer coupled to the digital server analyzes any changes in the infected surface areas and records those changes and the time interval between the images to determine a rate of change. The changes and rate of change allow an evaluation to be performed in a digital processor to determine an inhibition effectiveness of the inhibition of the microbial growth of the embodiment of a terpene isolates concentration of one embodiment.

Terpenes to be evaluated by anti-microbial category for fungi include eugenol, geraniol, alpha-bisabolol, alpha-terpineol, caryophyllene oxide, nerolidol, and ocimene. Terpenes to be evaluated for anti-bacterial activity include thymol, citral, limonene, linalool, caryophyllene oxide, and nerolidol. Terpenes to be evaluated for activity against Mites/Aphids include terpinolene, para-cymene, and ocimene.

The method of determining terpene-based biocontrol agents for use on cannabis and hemp include processing steps that include a. Isolate epiphytic microorganisms from cannabis ‘flower wash’ and isolate into pure cultures. b. Grow up individual fungal or bacterial broth cultures with shaking at RT until turbidity is evident; if, possible take OD_{600 nm} reading of turbidity for each culture just prior to plating out serial dilutions onto agar plates to determine ˜CFU/ml. c. Based on CFU data from (b) above, re-inoculate fresh broth cultures for individual cultures monitoring OD_{600 nm} to reach previous data point and make appropriate dilutions to attain complete seeding on agar petri plate. d. While allowing bacterial suspension to be absorbed into agar, prepare terpene-treated Whatman filter paper hole punches by aliquoting 25 μl of either full strength, 1:10 dilution or 1:100 dilution. Dilutions should be made into 1-15% MeOH or 1-2% castile soap. e. Place terpene-soaked discs in a pattern onto the bacterial inoculated plates in duplicates with a diluent-soaked negative control disc in the center as seen in FIG. 1. i. One terpene per plate at three concentrations with negative control. ii. One microbial culture seeded per plate. f. Allow plates to grow with incubation at 28° C. for overnight and photograph as well as measuring any zones of inhibition. Continue incubation for 2 more days, taking photographs and making measurements after each 24-hr period. g. Repeat steps (c) through (f) for each terpene against all microbial cultures.

An optical camera and an infrared camera with Wi-Fi and cellular connectivity coupled electronically to a digital server using cellular connectivity captures time-stamped images at intervals of individual cultures during incubation. The images are analyzed for measuring any zones of inhibition and the measurement data is recorded with the time interval between images to determine the changes in the zones of inhibition and a corresponding rate of change is calculated using a digital processor coupled to the digital server and server computer. The data was further evaluated to determine the effectiveness of the terpene-based biocontrol agents treatments.

Sensors coupled to the digital server computer record data for detected temperatures, and changes in density of the incubated microorganisms using distances sensed from the top surface of the microorganisms to the surface of the culture plate surface. Chemical sensors perform chemical analysis of vapors being released during the incubation heating process to detect any changes in the vapor composition to identify components of the microorganisms being inhibited.

The testing personnel may log in to the digital server via Wi-Fi or cellular capable digital devices including laptop computer, smart phone, tablets and other digital device to review the inhibition data being collected. The digital server will send Wi-Fi and cellular alerts to the testing personnel should the data being collected register any abrupt or abnormal changes that are outside a calculated trend line of the progression of the inhibition data being gathered and recorded of one embodiment.

1. Test combined bioactive terpenes identified under Specific Aim #1 above for synergy. 2. Develop terpene formulations and retest as in Specific Aim #1 to ensure that added ingredients do not interfere or dampen bioactivity. 3. Test terpene formulations on cannabis seedlings for suitability. 4. Test terpene formulations in challenge experiments on cannabis seedlings. An initial timeframe to complete a battery of testing can for example be 9 months of one embodiment.

The data gathered from inhibition testing on living infected cannabis seedlings and plants and laboratory tested inhibition cultures are analyzed using digital processors of the digital server to determine any differences in the effectiveness of the inhibition responses between “field” testing living plants and cultures in a controlled laboratory setting. Any correlations developed between the two sets of data for example time intervals for inhibition of microbial growth, are then used to develop adjustments in concentration levels of particular terpenes to aid in the development of the terpene inhibition treatment formulations of one embodiment.

### Evaluation Results #1 of Individual Terpenes for Effectiveness:

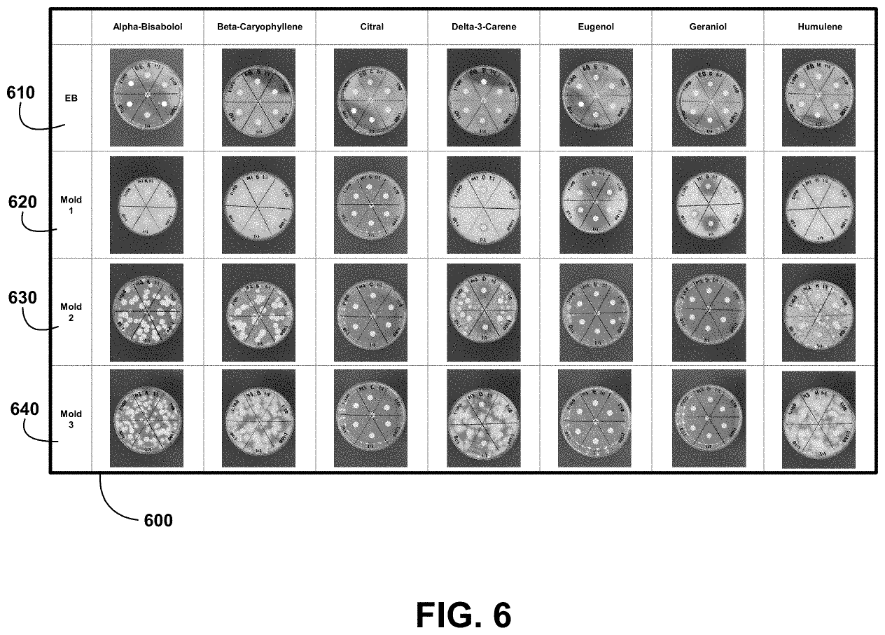

FIG. 6 shows for illustrative purposes only an example of evaluation results #1 of individual terpenes for effectiveness of one embodiment. FIG. 6 shows an example of evaluation results #1 of individual terpenes for effectiveness. The results are displayed in rows and columns. The rows are the EB Plate, Mold Plate 1, Mold Plate 2, and Mold Plate 3 in a FIG. 1 pattern. The terpene-soaked discs are identified for the particular terpene as indicated at the top of each column for example alpha-bisabolol in column 1 and beta-caryophyllene in column 2 and so forth including citral, delta-3-carene, eugenol, geraniol, and humulene. The effectiveness results show the dramatic differences with citral in Column 3 versus beta-caryophyllene in column 2 of one embodiment.

### Evaluation Results #2 of Individual Terpenes for Effectiveness:

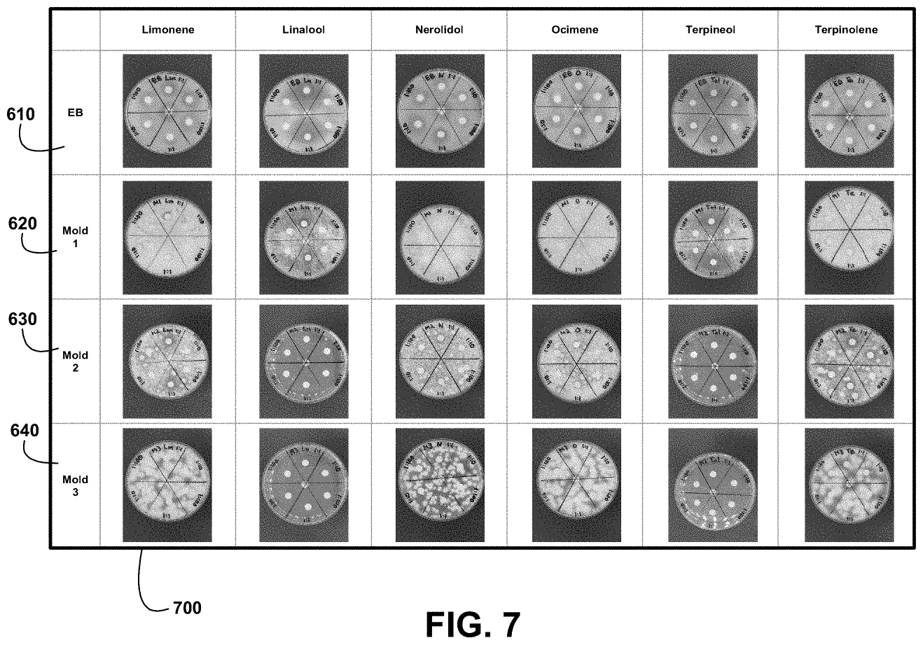

FIG. 7 shows for illustrative purposes only an example of evaluation results #2 of individual terpenes for effectiveness of one embodiment. FIG. 7 shows evaluation results #2 of individual terpenes for effectiveness for a different group of terpenes. The effectiveness results in evaluation results #2 again show the dramatic differences in terpene effectiveness as seen between limonene in column 1 versus linalool in column 2 and the other columns including nerolidol, ocimene, terpineol, and terpinolene of one embodiment.

### Terpinolene Concentrations Treatment Sample Culture Plates:

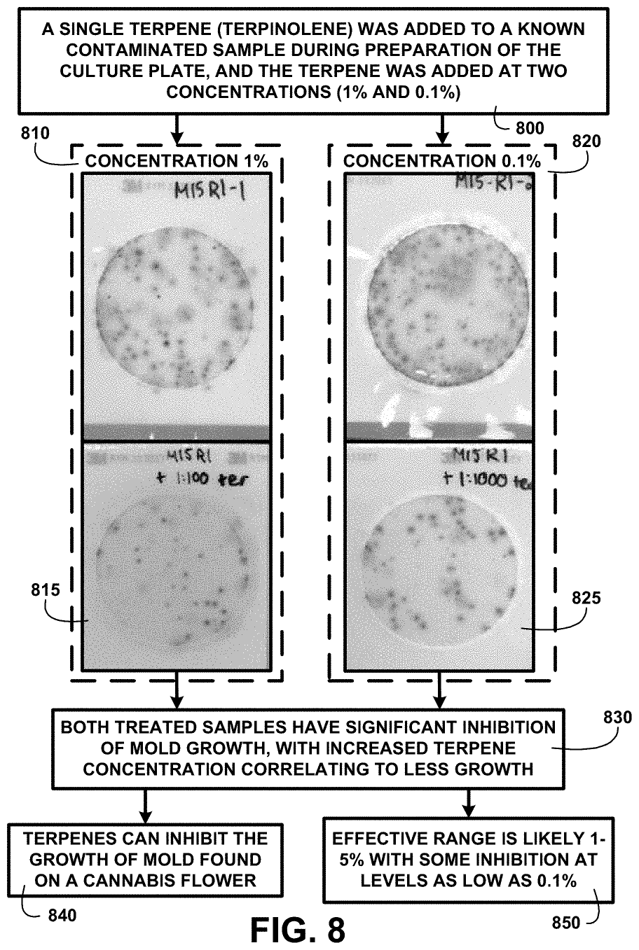

FIG. 8 shows for illustrative purposes only an example of terpinolene concentrations treatment sample culture plates of one embodiment. FIG. 8 shows a single terpene (terpinolene) was added to a known contaminated sample during preparation of the culture plate, and the terpene was added at two concentrations (1% and 0.1%) 800. A first culture plate with the concentration 1% 810 shows the concentration 1% inhibition 815. A second culture plate with the concentration 0.1% 820 shows the concentration 0.1% inhibition 825. Both treated samples have significant inhibition of mold growth, with increased terpene concentration correlating to less growth 830. Terpenes can inhibit the growth of mold found on a cannabis flower 840. An effective range is 1-5% with some inhibition at levels as low as 0.1% 850 of one embodiment.

Cultured Yeast and Mold Species from Certified Reference Materials:

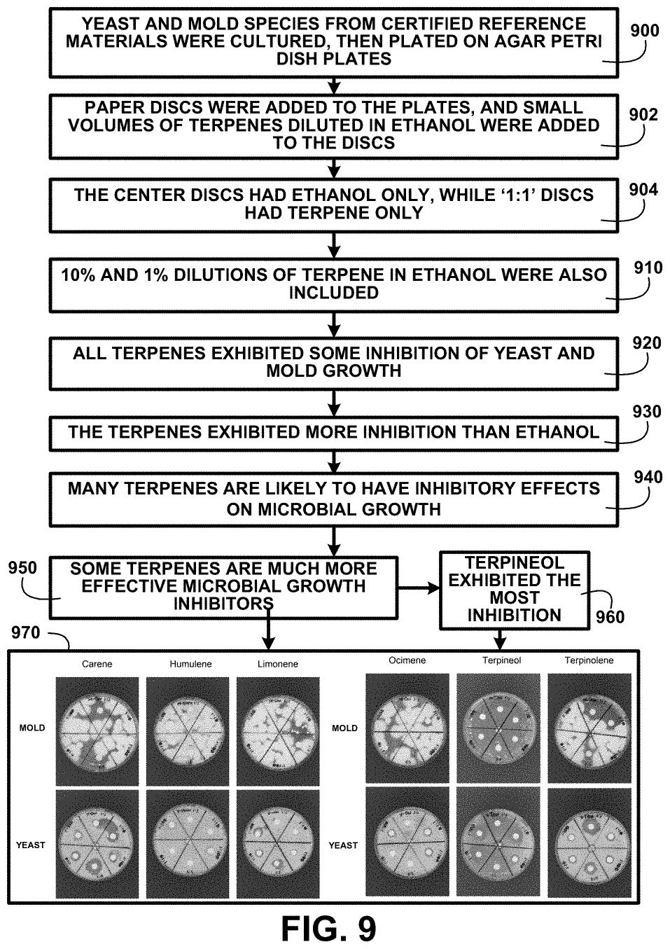

FIG. 9 shows for illustrative purposes only an example of cultured yeast and mold species from certified reference materials of one embodiment. FIG. 9 shows yeast and mold species from certified reference materials were cultured then plated on agar petri dish plates 900. Paper discs were added to the plates, and small volumes of terpenes diluted in ethanol were added to the discs 902. The center discs had ethanol only, while ‘1:1’ discs had terpene only 904 and 10% and 1% dilutions of terpene in ethanol were also included 910. All terpenes exhibited some inhibition of yeast and mold growth 920. The terpenes exhibited more inhibition than ethanol 930. Many terpenes are likely to have inhibitory effects on microbial growth 940. Some terpenes are much more effective microbial growth inhibitors 950. Terpinolene exhibited the most inhibition 960. Terpenes microbial growth inhibitor samples 970 include carene, humulene, limonene, ocimene, terpineol, and terpinolene of one embodiment.

### Several Microbial Species Cultures:

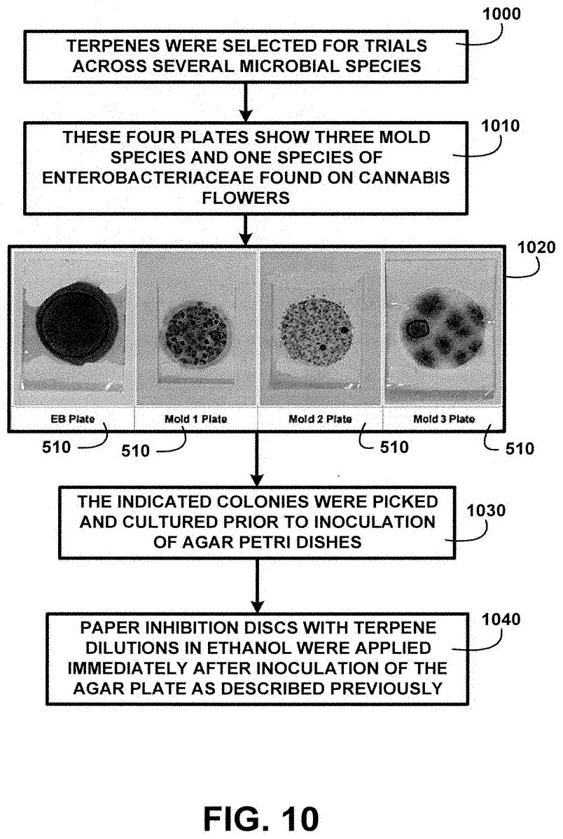

FIG. 10 shows for illustrative purposes only an example of several microbial species cultures of one embodiment. FIG. 10 shows terpenes were selected for trials across several microbial species 1000. These four plates show three mold species and one species of Enterobacteriaceae found on cannabis flowers 1010. One Enterobacteriaceae species sample plate and three mold species sample plates 1020. An Enterobacteriaceae Count Plate referred to herein as EB plate 1022, and mold species sample plates Mold 1 plate 1024, Mold 2 plate 1026, and Mold 3 plate 1028. The indicated colonies were picked and cultured prior to inoculation of agar petri dishes 1030. Paper inhibition discs with terpene dilutions in ethanol were applied immediately after inoculation of the agar plate as described previously 1040

### Group 1 Terpenes Selected for Trials Across Several Microbial Species:

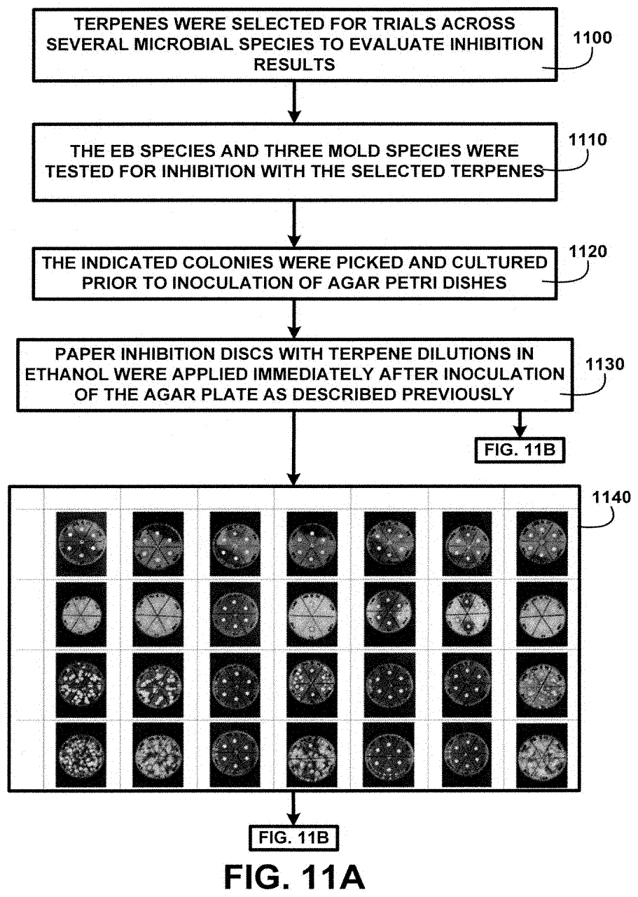

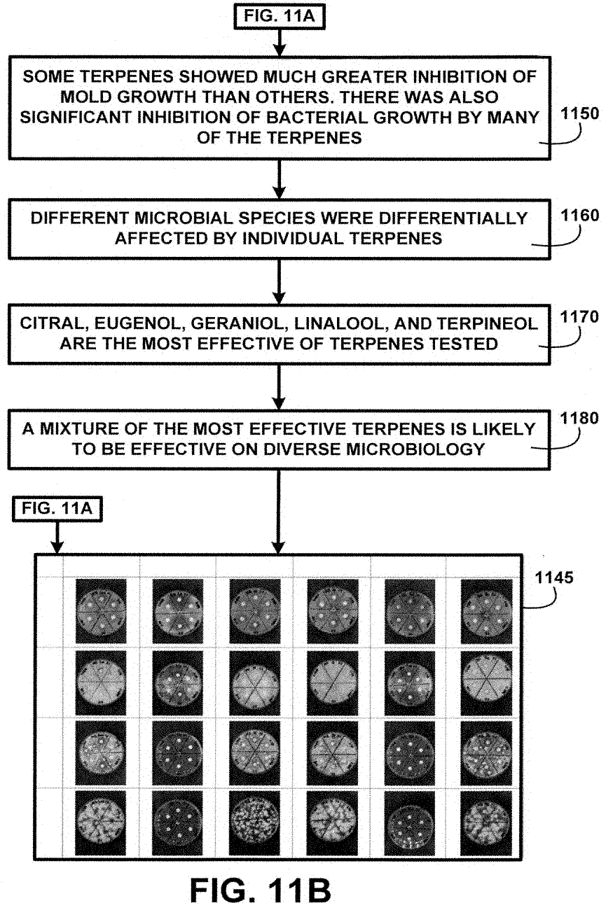

FIG. 11A shows for illustrative purposes only an example of group 1 terpenes selected for trials across several microbial species of one embodiment. FIG. 11A shows terpenes were selected for trials across several microbial species to evaluate inhibition results 1100. The EB species and three mold species were tested for inhibition with the selected terpenes 1110. The indicated colonies were picked and cultured prior to inoculation of agar petri dishes 1120. Paper inhibition discs with terpene dilutions in ethanol were applied immediately after inoculation of the agar plate as described previously 1130. FIG. 11A shows the terpenes selected for trials in group 1 paper inhibition discs with terpene dilutions in ethanol 1140 including alpha-bisabolol, beta-caryophyllene, citral, delta-3-carene, eugenol, geraniol, and humulene of one embodiment. Terpenes selected for trials in group 2 of are shown in FIG. 11B. The results of the trials across several microbial species is continued in FIG. 11B.

### Group 2 Terpenes Selected for Trials Across Several Microbial Species:

FIG. 11B shows for illustrative purposes only an example of group 2 terpenes selected for trials across several microbial species of one embodiment. FIG. 11B shows a continuation from FIG. 11A. Some terpenes showed much greater inhibition of mold growth than others. There was also significant inhibition of bacterial growth by many of the terpenes 1150. Different microbial species were differentially affected by individual terpenes 1160. Citral, eugenol, geraniol, linalool, and terpineol are the most effective of terpenes tested 1170. A mixture of the most effective terpenes is likely to be effective on diverse microbiology 1180. The results of the trials across several microbial species continued from FIG. 11A is shown in group 2 paper inhibition discs with terpene dilutions in ethanol 1145 including limonene, linalool, nerolidol, ocimene, terpineol, and terpinolene of one embodiment.

Cultures Isolated from Cannabis Flower:

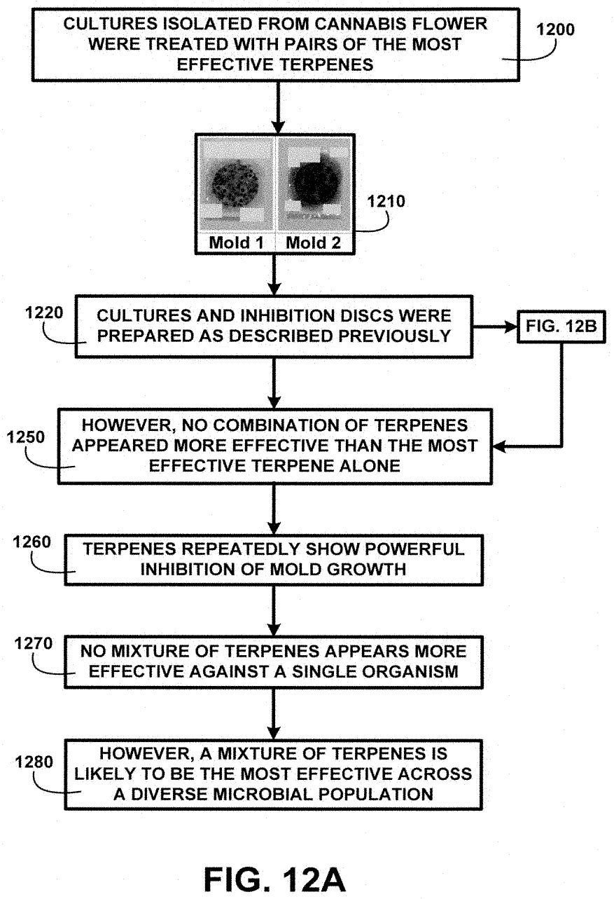

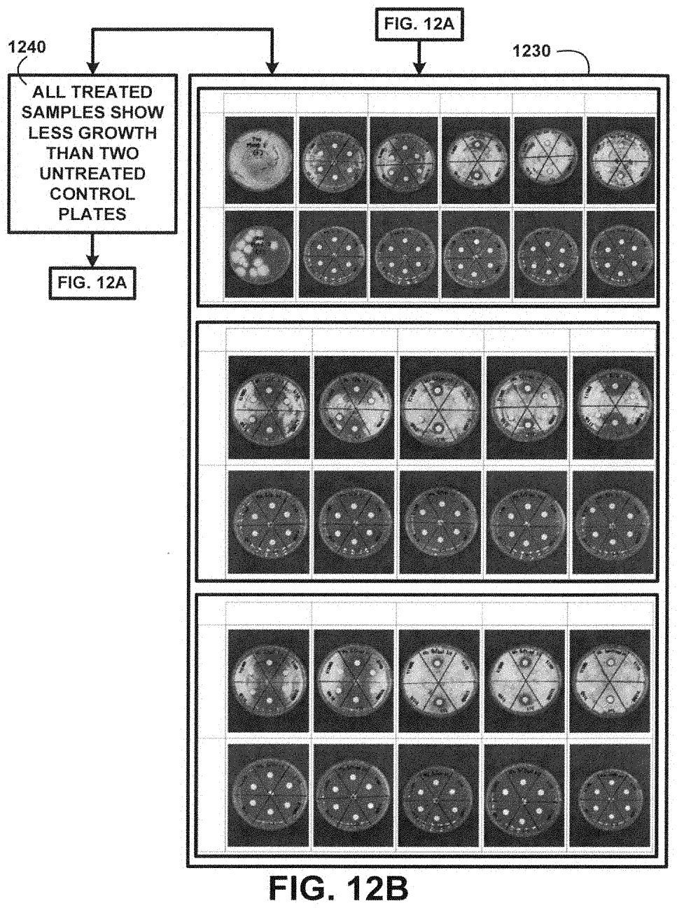

FIG. 12A shows for illustrative purposes only an example of cultures isolated from cannabis flower of one embodiment. FIG. 12A shows cultures isolated from cannabis flower were treated with pairs of the most effective terpenes 1200. The cultures isolated from cannabis flower 1210 are shown in the Mold 1 and Mold 2 culture plates. Cultures and inhibition discs were prepared as described previously 1220 and shown in FIG. 12B. The results shown in FIG. 12B show however, no combination of terpenes appeared more effective than the most effective terpene alone 1250. Terpenes repeatedly show powerful inhibition of mold growth 1260. No mixture of terpenes appears more effective against a single organism 1270. However, a mixture of terpenes is likely to be the most effective across a diverse microbial population 1280 of one embodiment.

### All Treated Samples Show Less Growth:

FIG. 12B shows for illustrative purposes only an example of all treated samples shows less growth of one embodiment. FIG. 12B shows a continuation from FIG. 12A including two positive control plates and cultures and inhibition discs of single terpenes including citral, delta-3-carene, eugenol, geraniol, linalool, and terpineol. FIG. 12B also shows cultures and inhibition discs show the effectiveness of the various combinations of terpenes 1230 including citral/eugenol, citral/geraniol, citral/linalool, citral/terpineol, eugenol/geraniol, eugenol/linalool, eugenol/terpineol, geraniol/linalool, geraniol/terpineol, and linalool/terpineol. All treated samples show less growth than two untreated control plates 1240. The results shown in FIG. 12B are evaluated further in FIG. 12A of one embodiment.

### Remediation by Terpene Treatment:

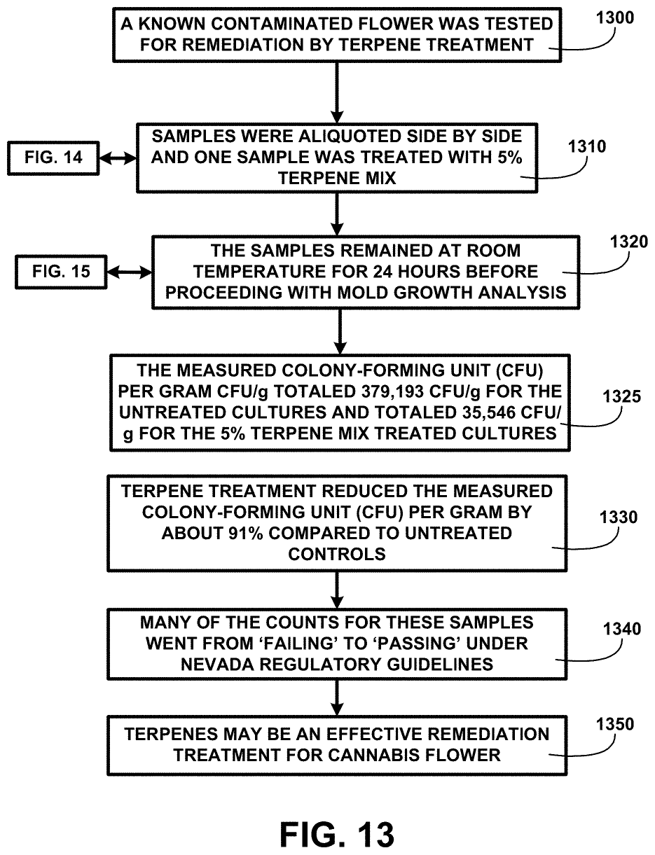

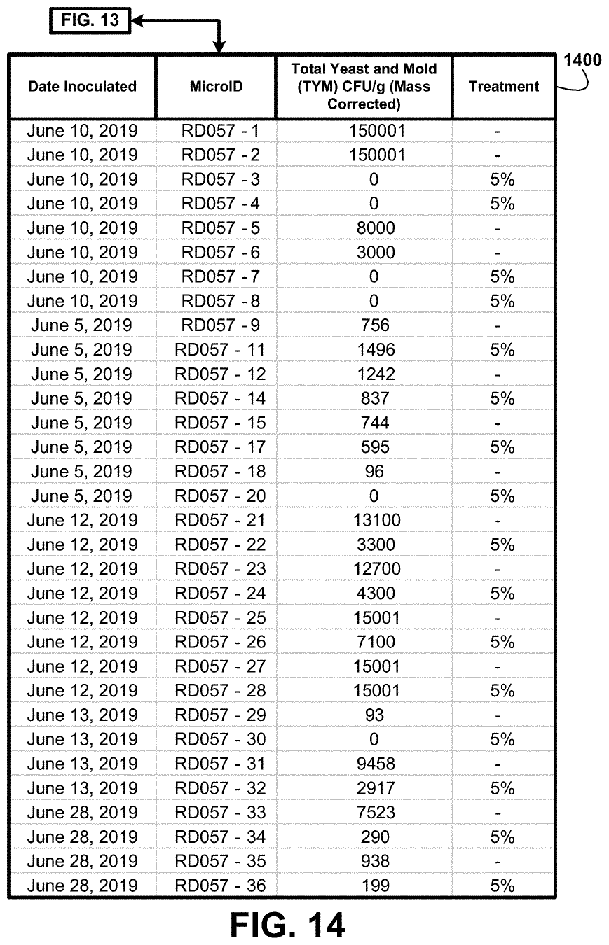

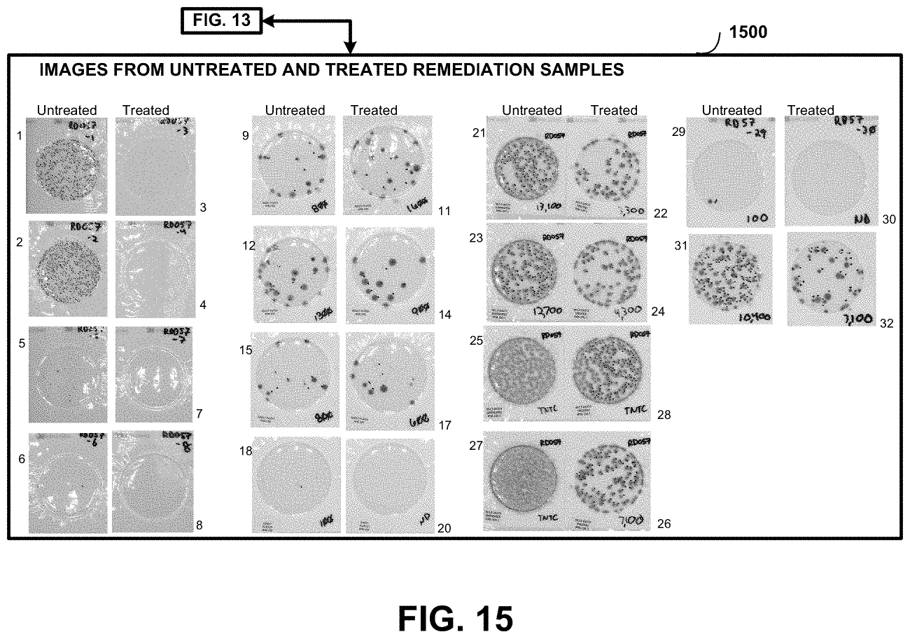

FIG. 13 shows a block diagram of an overview of remediation by terpene treatment of one embodiment. FIG. 13 shows a known contaminated flower was tested for remediation by terpene treatment 1300. Samples were aliquoted side by side and one sample was treated with 5% terpene mix 1310. The results are shown in FIG. 14. The samples remained at room temperature for 24 hours before proceeding with mold growth analysis 1320 as shown in FIG. 15. The mold growth analysis results show the measured colony-forming unit (CFU) per gram CFU/g totaled 379,193 CFU/g for the untreated cultures and totaled 35,546 CFU/g for the 5% terpene mix treated cultures 1325. The mold growth analysis results show the terpene treatment reduced the measured colony-forming unit (CFU) per gram (CFU/g) by about 91% compared to untreated controls 1330. Many of the counts for these samples went from ‘failing’ to ‘passing’ under Nevada regulatory guidelines 1340. Terpenes may be an effective remediation treatment for cannabis flower 1350 mold infections of one embodiment.

### Total Yeast and Mold CFU/q Remediation:

FIG. 14 shows a block diagram of an overview of a tabulation of remediation by terpene treatment mold growth analysis results of one embodiment. FIG. 14 shows a continuation from FIG. 13 with a tabulation of results from a remediation by terpene treatment testing method 1400. The tabulation shows an Untreated Yeast and Mold cultures mold growth analysis for a measure of viable bacterial or fungal cells prior to inhibition inoculation with a 5% terpene mix. The measure of viable bacterial or fungal cells as solids and calculated by colony-forming units per gram (CFU/g). The Untreated Yeast and Mold cultures are identified by a “-” symbol in the Treatment column. The tabulation also shows the CFU/g of the corresponding Treated cultures after inoculation. The Treated cultures are identified by the “5%” entry in the Treatment column. The 5% entry corresponds to the terpenoid concentration (%) used to inoculate the Untreated Yeast and Mold cultures.

The tabulation shows the measured colony-forming units per gram for the Untreated Yeast and Mold cultures totaling 379,193 CFU/g. The tabulation also shows the measured colony-forming units per gram for the Treated cultures using the terpenoid 5% concentration for inoculation totaling 35,546 CFU/g. The terpene treatment reduced the measured colony-forming unit (CFU) per gram by about 91% compared to untreated controls 1330 of FIG. 13. The following results Jun. 28, 2019, RD057-33, 7523, -; Jun. 28, 2019, RD057-34, 290, 5%; Jun. 28, 2019, RD057-35, 938, -; and Jun. 28, 2019, RD057-36, 199, 5%; were not included in the tabulation of the findings and the corresponding Untreated and Treated cultures are not shown in FIG. 15.

Images from Untreated and Treated Remediation Samples:

FIG. 15 shows for illustrative purposes only an example of images from untreated and treated cultures remediation samples of one embodiment. FIG. 15 shows from FIG. 13 images from untreated and treated remediation sample culture plates 1-32 1500 of Untreated Yeast and Mold cultures and Treated cultures of the remediation by terpene treatment testing for inhibition inoculation with a 5% terpene mix. Culture plates 1-32 samples were aliquoted side by side and one sample was treated with 5% terpene mix 1310 of FIG. 13. The 5% terpene mix treated culture plates show the effectiveness of the inhibition inoculation remediation by terpene treatment of one embodiment.

### Digital Inhibition Evaluation Device:

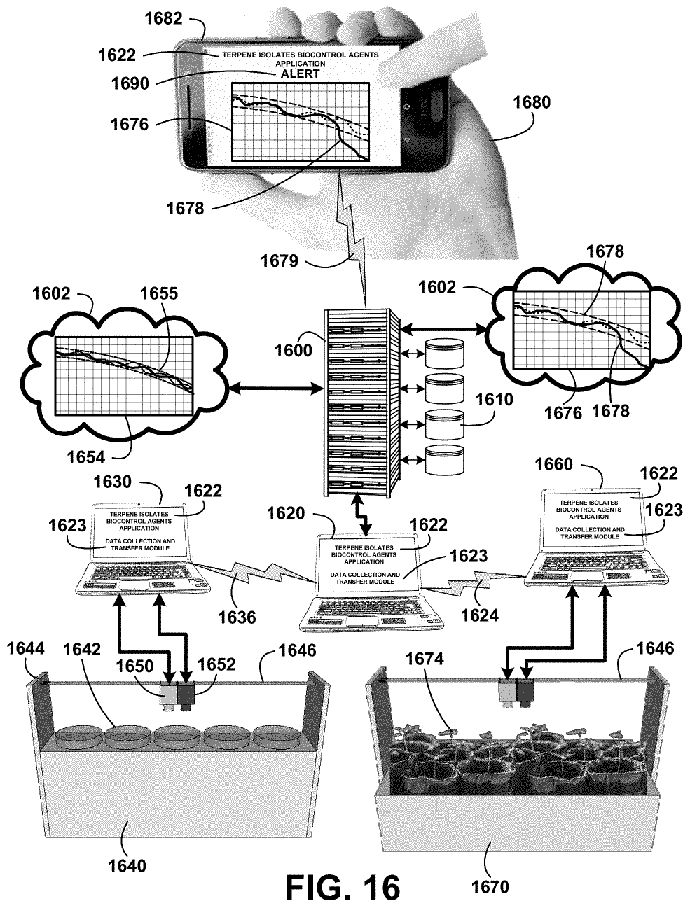

FIG. 16 shows for illustrative purposes only an example of a digital inhibition evaluation device of one embodiment. FIG. 16 shows a digital server 1600 coupled to a plurality of databases 1610. The digital server 1600 includes at least one digital server AI cloud 1602. Coupled to the digital server 1600 is at least one digital server computer 1620. The at least one digital server computer 1620 has installed a terpene isolates biocontrol agents application 1622 with at least one data collection and transfer module 1623. The at least one digital server computer 1620 at least one data collection and transfer module 1623 receives and transmits data collected to the digital server 1600 through direct cabling in one embodiment and by wireless transmission in another embodiment.

A lab testing plate structure 1640 supports a plurality of petri dish inhibition sample plates 1642. A camera rail support and motorized travel device 1644 is positioned at two ends of the lab testing plate structure 1640 to support and operate camera travel worm drive devices 1646.

An optical camera 1650 and infrared camera 1652 are coupled to the camera travel worm drive devices 1646 for positioning the cameras over each petri dish inhibition sample plates 1642. At least one sensor is coupled to the optical camera 1650 and infrared camera 1652 for detecting environmental and inhibition growth conditions and by-products. Inhibition growth conditions may include temperature, humidity, light intensity and others. Inhibition growth by-products may include chemical sensors for performing chemical analysis of by-product vapors being released during the inhibition testing to detect any changes in the vapor composition for identifying components of the microorganisms being testing for inhibition.

The data being collected during the lab testing is transmitted from the cameras and sensors and transmitted to the lab computer 1630. The terpene isolates biocontrol agents application 1622 categorizes the data in a predetermined format for wireless lab computer transmission of data 1636 to the digital server computer 1620. The optical camera 1650 may include a depth sensing camera for capturing 3D images of the lab growth cultures and cannabis seedlings. The optical camera 1650 and infrared camera 1652 include zoom features for close up views, tilt features for angling the view, and augmented lighting sources to adjust the viewed object lighting effects for example lessening shadows which can be adjusted by a user using the terpene isolates biocontrol agents application 1622 and for viewing the camera images on a user digital device with a display for example a smart phone, tablet or laptop computer.

A field seedling growing structure 1670 supports the cannabis seedling 1674 growth containers. The camera rail support and motorized travel device 1644 is placed at two ends of the field seedling growing structure 1670 to support and operate the camera travel worm drive devices 1646. An optical camera 1650 and infrared camera 1652 are coupled to the camera travel worm drive devices 1646 to position the cameras over cannabis seedlings. A depth sensing optical camera 1650 capturing 3D images of a seedling allows the user to inspect the seedling surfaces which vary in height and width. Not shown are reflective mirrored devices positioned at soil levels beneath the seedlings. Using the tilt features of the cameras to view the reflective mirrored devices a user may view the cannabis seedling leaves underneath surfaces for analysis of microorganisms on those surfaces. The field testing data is transmitted wirelessly to a field testing computer 1660. The field testing computer 1660 has installed the terpene isolates biocontrol agents application 1622 with the data collection and transfer module 1623. The terpene isolates biocontrol agents application 1622 categorizes the data in a predetermined format for wireless field testing computer transmission of data 1624 to the digital server computer 1620.

The digital server computer 1620 using a plurality of digital processor coupled to the at least one digital server AI cloud 1602 perform an analysis of the collected lab and field testing data. The at least one digital server AI cloud 1602 calculates an inhibition results trend line with high and low range limit lines 1676. A lab testing inhibition analysis graph 1654 and a field testing inhibition analysis graph 1676 are created using the at least one digital server AI cloud 1602. The current lab sample and field testing cannabis seedling being observed results are superimposed on the inhibition results trend line with high and low range limit lines 1676.

The two graphs may be viewed by testing personnel 1680 by logging into the terpene isolates biocontrol agents application 1622 on a testing personnel digital device 1682. The digital server 1600 automatically transmits an abrupt or abnormal change 1678 in results falling outside the inhibition results trend line with high and low range limit lines 1676 in an alert 1690 to the terpene isolates biocontrol agents application 1622 on testing personnel digital device 1682. The testing personnel may review the details of the data collected using the terpene isolates biocontrol agents application 1622 to investigate possible causes for the change for example a rise or drop in temperature. The at least one digital server AI cloud 1602 additionally analyzes the data collected from sensors and creates graphical representations of that data.

The testing personnel using the terpene isolates biocontrol agents application 1622 may access each of those graphical representations to quickly determine potential causes. The data may show more than one abrupt change in different sensor data which could lead to a concerted causal effect. The digital server 1600 including the coupled devices and use of the terpene isolates biocontrol agents application 1622 form a terpene isolates biocontrol agents network for controlling, monitoring and analyzing the laboratory and field testing of the method of determining terpene-based biocontrol agents for cannabis and hemp of one embodiment.

The foregoing has described the principles, embodiments and modes of operation of the embodiments. However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1.-10. (canceled)

11. An apparatus, comprising:
at least one device for isolating individual terpene isolates from plant derived essential oils;
a terpene isolates biocontrol agents network for controlling, monitoring and analyzing the laboratory and field testing;
at least one device coupled to the digital server for performing remediation by terpene isolates treatment mold growth analysis on untreated microbial species cultures and terpene isolates treated cultures and for calculating and displaying the mold growth analysis results;
at least one device for developing effective terpene isolates formulations for various microbial species using data recorded on the digital server;
at least one device for mixing formulated individual terpene isolates concentrations and for mixing formulated combinations of individual terpene isolates concentrations;
at least one device for applying terpene isolates formulations on cannabis seedlings and plants; and
a terpene isolates biocontrol agents network for controlling and analyzing the laboratory and field testing.

12. The apparatus of claim 11, further comprising at least one device for isolating epiphytic microorganisms from cannabis ‘flower wash’ into pure cultures;

13. The apparatus of claim 11, further comprising at least one sensor, at least one communication device with Wi-Fi and cellular connectivity, at least one digital optical camera with Wi-Fi and cellular connectivity, at least one infrared camera with Wi-Fi and cellular connectivity, at least one scanner with OCR and at least one printer coupled to the at least one digital server with a computer for monitoring and analyzing at least one terpene isolates treatment mold growth analysis.

14. The apparatus of claim 11, further comprising at least one device for correlating data from field testing and laboratory culture testing for inhibition of microbial growth to aid in developing terpene isolates inhibition treatment formulations.

15. The apparatus of claim 11, further comprising at least one device for performing predetermined incubation of untreated microbial species cultures and for predetermined incubation of terpenoid concentrations treated cultures.

16. An apparatus, comprising:
a terpene isolates biocontrol agents network for controlling, monitoring and analyzing the laboratory and field testing growth inhibition results of terpene isolates treatment of microbial species on cannabis plants and cultures;
at least one device for performing remediation by terpene isolates treatment mold growth analysis and for calculating and displaying the mold growth analysis results; and
at least one device for correlating data gathered from field testing and laboratory culture testing for inhibition of microbial growth for developing terpene inhibition treatment formulations.

17. The apparatus of claim 16, wherein remediation by terpene isolates treatment mold growth analysis determines a measure of viable bacterial or fungal cells after an inhibition inoculation with at least one terpene isolate.

18. The apparatus of claim 16, further comprising remediation by terpene isolates treatment mold growth analysis is used for determining a measure of viable bacterial or fungal cells before an inhibition inoculation with at least one terpene isolate for use in determining the effectiveness of the measure of viable bacterial or fungal cells after an inhibition inoculation with at least one terpene isolate.

19. The apparatus of claim 16, further comprising at least one digital optical camera with Wi-Fi and cellular connectivity, at least one infrared camera with Wi-Fi and cellular connectivity, and sensors coupled to the at least one digital server with a computer for gathering terpene isolate treatment microbial species growth inhibition data for use in determining the effectiveness of the terpene isolate treatment microbial species growth inhibition.

20. The apparatus of claim 16, wherein the at least one device for correlating data includes processing at least measurements of viable bacterial or fungal cells as solids and calculated by colony-forming units per gram (CFU/g), optical camera time-stamped images and infrared camera time-stamped images of microbial species growth inhibition for terpene isolates inoculation inhibition treatments, sensor temperature readings, microorganism densities sensor readings, and sensor chemical analysis of terpene isolates inoculation inhibition treated culture vapor emissions from field testing and laboratory culture testing.
